(19) United States
(12) Patent Application Publication
(10) Pub. No.: US 2014/0276242 A1
(43) Pub. Date: Sep. 18, 2014

(54) WEARABLE BODY 3D SENSOR NETWORK SYSTEM AND METHOD

(71) Applicant: Healthward International, LLC, Plano, TX (US)

(72) Inventors: Shyh-Min Chen, Plano, TX (US); Manli Yang, Fort Worth, TX (US); Arthur Tu, Plano, TX (US)

(73) Assignee: Healthward International, LLC, Plano, TX (US)

(21) Appl. No.: 14/155,912

(22) Filed: Jan. 15, 2014

Related U.S. Application Data
(60) Provisional application No. 61/784,123, filed on Mar. 14, 2013.

(51) Int. Cl. A61B 5/11; A61B 5/103

(52) U.S. Cl. CPC A61B 5/1126; A61B 5/1038; A61B 5/112
USPC 600/595

(57) ABSTRACT

The disclosed principles provide wearable body sensor systems and related methods to evaluate the whole body movement with or without an assistive walking device. More specifically, the disclosed principles monitor the whole body movement including COG & BOS with a 3-dimensional (3D) mapping over a given period of time. The disclosed principles provide a number of objectives, which include providing a wearable body 3D sensor network that can constantly monitor change of COG over the BOS during daily mobility; to clinically evaluate older adults in maintaining upright posture and prevent falls in senior living communities; and to clinically establish a quantitative mathematical model to determine the stability of the older adults.

### PRIORITY CLAIM

The present disclosure is a non-provisional conversion of, and thus claims priority to, U.S. Provisional Patent Application No. 61/784,123, filed Mar. 14, 2013, which is herein incorporated by reference in its entirety for all purposes.

### TECHNICAL FIELD

The disclosed principles relate generally to body movement and posture evaluation, and more particularly to a novel 3D sensor network for mapping body movement and posture.

### BACKGROUND

Gravity plays an essential role in pulling people down to the ground (earth). Consequently, humans fight against gravity most fiercely in the early stage of life, e.g., as a toddler, as well as in the later stage, e.g., as an older or elderly adult. For older adults to maintain independent living, it is critical that they need to demonstrate ability to walk with upright posture with or without an assistive device such as a cane.

Leaning posture and unstable gait are very common reasons that may cause balance deficits and frequent falls in older adults. Moreover, accidental falls are the leading cause of death in people who are 65 years and older. Typically, one supposes to walk in an upright posture, which makes the person's center of gravity (COG) fall perpendicularly (plummet line) on the center of the person's base of support (BOS). The COG for a person is normally at the level of the 2^{nd} sacral vertebra at the upright posture. The BOS for a person is the area bounded by both feet in contact with ground/floor when a person is standing with both feet, or the area just bounded by the foot, when one is standing on a single foot.

During standing or walking, a person has to maintain his/her COG constantly and consistently within the BOS. However, an unstable walking pattern or posture problem, such as leaning forward or sideways, could change a person's COG and result in deviation of the COG plummet line away from the center of the BOS. When the deviation is too much, the fall will consequently occur.

The human body is an integrated system that is interconnected and interacted by many joints at head/neck, shoulder, trunk, hip, knee, and ankle locations. These joints work together to maintain a person in upright posture and stable gait. When one joint becomes weak, other joints will typically compensate. Practically, the more a joint plays a compensatory role, the more possible the body posture and gait will change. When an assistive walking device, such as walking cane or stick, is used, the COG & BOS will be changed for a human body in order to gain balance during standing and walking

Despite the need for systems and methods to monitor the posture and gait of a person, conventionally available body sensor systems do not monitor the entire body movement of a person, including monitoring the person's COG & BOS. For example, FIG. 1 sets forth a diagram 100 illustrating a conventional sensor 105 for measuring joint movement of a wearer's arm 110. Specifically, exemplary mechanical trackers that have been utilized include rigid or flexible goniometers 105, which are angular measuring devices worn by the user. These angular measuring devices provide joint angle data to kinematic algorithms that are used to determine body posture. However, problems with employing conventional mechanical trackers include their use with the soft tissue of the body, and more specifically that the soft tissue typically allows the position of the linkages relative to the body to change as motion occurs. Such unwanted movement results in less accurate movement tracking Moreover, beyond such movement during use, alignment of such goniometers with body joints is typically difficult, especially use on joints having multiple axes of movement.

Even more generally, such mechanical tracking devices are limited to providing data on the movement of the joint they are connected to, and are not capable of providing movement information in relation to the movement of other joints and locations across the wearer's body, as well as movement and position of the wearer's body as a whole. Monitoring of the whole body movement is also a crucial evaluation tool when an assistive walking device is used, and such limited mechanical trackers cannot provide data on such assistive walking devices. Accordingly, what is needed in the art is a system and method for monitoring and evaluating the whole body movement of a person, with or without a walking device that does not suffer from the deficiencies of conventional approaches.

### SUMMARY

The disclosed principles provide wearable body sensor systems and related methods to evaluate the whole body movement with or without an assistive walking device. More specifically, the disclosed principles monitor the whole body movement including COG & BOS with a 3-dimensional (3D) mapping over a given period of time. The COG and BOS of a person are dynamic as the person moves, and thus the disclosed principles provide a number of objectives, which include providing a wearable body 3D sensor network that can constantly monitor change of COG over the BOS during daily mobility to clinically evaluate older adults in maintaining upright posture and to help prevent falls in senior living communities and to clinically establish a quantitative mathematical model to determine the stability of the older adults. More specifically, in advantageous embodiments, a wearer of the body sensor network (e.g., a patient) may have a baseline or threshold established by having their posture, gait, or other body positions and movements monitored and recorded using the sensor network. That baseline data may then be stored and compared against the wearer's later body positions and movements to detect degradation in one or more positions or movements over time, and additionally detect potentials for falls or other detrimental results of the wearer's current and ongoing body positioning and movement.

Exemplary implementations may include use for an older adult whose body position or gait unconsciously continues to or increasingly walks in a leaning-forward posture during daily mobility with or without using a cane or a walker. The disclosed sensor technique would be able to alert the sensor user to straighten his body up as a preventative measure to protect the body from developing a kyphotic (leaning-forward) posture. Moreover, the sensor network and system may alert caretakers, doctors, etc. of the situation as it occurs, and even predict a continual degradation. In other implementations, a user of a disclosed sensor system may turn his head (a head/neck movement on an axial plane) and as a result fall on the floor without being seen by others. The data from the system can be used for data tracking to see which joint initiated the chain-reaction that led to the fall. In yet another exemplary implementation, for a person who is receiving interventions for balance or gait deficiencies, a system in accordance with the disclosed principles is able to monitor how the body progresses in terms of joints and joint movements using 3D mapping.

In one particular embodiment, a system constructed in accordance with the disclosed principles is configured for evaluating movement of a human body, and may comprise a central sensor node positioned substantially at the center of gravity of the body. In addition, such a system may also comprise a network of sensors dispersed about predetermined locations on the body, and configured to gather data regarding positions of the predetermined locations on the body during movement of the body's center of gravity with respect to the body's base of support. Such system may further comprise a computing device configured to receive sensor data from the central sensor node and the network of sensors. Based on the received data, the computing device may be configured to detect balance deficiencies of the body based on positions of the body's center of gravity in relation to its base of support during movement of the body, as well as evaluate the gathered data to determine unfavorable positions of one or more of the predetermined locations during detected balance deficiencies. Still further, exemplary embodiments of the disclosed system may also include a feedback device configured to provide feedback when one or more of the predetermined locations of the body moves into one or more corresponding unfavorable positions. The feedback may be provided to the wearer, to personnel tasked with monitoring the well-being of the wearer, or both.

In other aspects, methods for evaluating movement of a human body in accordance with the disclosed principles are also disclosed herein. In one embodiment, such a method may comprise detecting the body's center of gravity using data gathered from a central sensor. Such a method may further comprise gathering data from a network of sensors positioned at predetermined locations on the body during movement of the body's center of gravity with respect to the body's base of support. In such embodiments, the method may also include detecting, based on the data gathered from the central sensor and the network of sensors, balance deficiencies of the body based on positions of the body's center of gravity in relation to its base of support during movement of the body. In addition, such methods may also include evaluating the gathered data to determine unfavorable positions of one or more of the predetermined locations during detected balance deficiencies. Moreover, such exemplary methods may also include providing feedback when one or more of the predetermined locations of the body moves into one or more corresponding unfavorable positions.

### BRIEF DESCRIPTION OF THE DRAWINGS

Exemplary embodiments of the disclosed principles are described herein with reference to the following drawings, in which like numerals identify similar components, and in which:

FIG. 1 illustrates a conventional sensor for measuring joint movement of a wearer's arm;

FIG. 2 illustrates a conceptual drawing of one embodiment of the layout of a 3D body sensor network that may be provided in a system or method in accordance with the disclosed principles;

FIG. 3 illustrates a block diagram setting forth the internal components of one embodiment of a sensor node in accordance with the disclosed principles;

FIG. 4 illustrates a block diagram setting forth one embodiment of the interconnection of a central sensor node with an external computing device;

FIG. 5 illustrates a block diagram setting forth the internal components of one embodiment of a bottom sensor node in accordance with the disclosed principles;

FIG. 6 illustrates a block diagram of the exemplary central sensor node of FIG. 4 with optional GPS tracking information;

FIG. 7 illustrates a diagram of a wearer of a sensor network as disclosed herein, where the specific locations of the sensor nodes are selected for providing such identification;

FIG. 8 illustrates exemplary locations for pressure sensors that may be included in each of the wearer's feet;

FIG. 9 illustrates how the center of gravity of a wearer changes as the wearer changes his positions;

FIGS. 10A-10D illustrate four examples of four different standing positions of a wearer of disclosed sensor network;

FIG. 11 illustrates the relationship of the COG angle as determined from the detected COG and BOS provided by the disclosed sensor network;

FIGS. 12A-12C illustrate plots, measured in inches, of an exemplary wearer's COG over BOS in varying stages of stability; and

FIG. 13 illustrates a flow diagram setting forth an exemplary process for determining and predicting the deteriorating gait a wearer for a sensor network as disclosed herein.

### DETAILED DESCRIPTION

As introduced above, the disclosed principles provide 3D body sensor systems and methods for prevention and intervention for persons having balance deficits consequently resulting in injuring falls. Such a 3D network makes it possible to capture real-time human body movement and posture in an ambulatory situation without the need of mechanical goniometers or external cameras. Initially captured data may be compared against later captured to detect and predict deteriorating body positions and movements. In exemplary embodiments, the disclosed principles utilize semiconductor based microelectromechanical system (MEMS) 3D inertial sensors to capture the movement of joint-based body parts/locations and dynamic change of posture during ambulation for the purpose of motion analysis. Such MEMS inertial sensors combine the signals from 3D gyroscopes, 3D accelerometers, and 3D magnetometers (compass sensors), in addition to other components, to provide the data employed by systems and methods of the disclosed principles. Of course, other types of sensor technology, either now existing or later developed, may also be employed with the novel 3D body mapping techniques disclosed herein.

Looking in particular at how such exemplary components work with the disclosed principles, gyroscopes are used to measure angular movement, and accelerometers are used to determine the direction of the local vertical by sensing acceleration due to gravity. In addition, magnetic sensors provide stability in the horizontal plane by sensing the direction of the earth magnetic field, such as with a compass. The disclosed principles may also utilize weight sensors to measure the weight pressure on each foot, as well as measure the weight pressure on an assistive walking device if such a walking device is used. The 3D sensor data and the weight sensor data captured through the sensor nodes throughout the user's body may then be used to evaluate the patient's full body movement, posture COG and BOS to determine whether a user has balance deficiencies. As noted above, although exemplary semiconductor MEMS sensors are discussed herein, any type of sensors that provide the same or equivalent data may also be implemented with the disclosed principles, and the present disclosure is not limited to any particular type of sensor.

Looking now at FIG. 2, illustrated is a conceptual drawing of one embodiment of the layout of a 3D body sensor network 200 that may be provided in a system or method in accordance with the disclosed principles. The inertia sensors for use in establishing a network that generates a 3D mapping of the wearer's movements as disclosed herein are wearable either directly on the skin, on the wearer's clothing, or even with dedicated garments having the sensors. With wearable sensors, body movements, posture, and position can be accurately tracked and represented to generate the 3D body movement mapping disclosed herein.

The tracking provided by the disclosed principles may be implemented for any activity in which body position and movement are important, such as remote patient monitoring, individualized instruction and outcome evaluation for rehabilitation, and other health applications. Moreover, the accurate detection and remote tracking of the disclosed principles may be used to track and evaluate patient movement and posture assessment cost effectively. In addition, the disclosed principles can provide for onscreen display of a patient's body movement, posture change, COG, and BOS during the wearer's monitored movements and activities. Such an onscreen display of the patient's movement, posture change, COG, and BOS can be on a real-time basis or can be based on historical data that was captured and stored, for example, in a central sensor unit or in a database. A comparison of real-time data with historical data may be used to detect deficiencies in position or movement, such as for detecting and predicting deteriorating posture and possible falls.

The disclosed sensor network 200 is worn by a user for body motion capturing, and gathers data regarding the user's body position and movement to generate a 3D mapping of that movement or posture for evaluation as disclosed herein. As illustrated in FIG. 2, sensor nodes 205 may be dispersed at select locations of the wearer's body 210, as well as their assistive walking device 215, if present. The locations selected for providing sensors are those locations of a human body that will provide the best and most accurate information regarding a body's movements. As illustrated, such locations may include the COG of a person at the center of the body's core. The COG and BOS are dynamic data. The constantly changing COG has to be maintained within constantly changing BOS to maintain wearer's stability. Thus, the disclosed principles calculates the COG & BOS on a real-time basis based on the feedback from the network of 3D sensors and weight sensors located throughout strategic positions on the body. The COG over BOS will then be evaluated on a real-time basis to evaluate the deficiency in position or movement of the wearer.

Such positioning for the central sensor at the wearer's COG is important for detecting changes in the wearer's COG over his BOS, using the central sensor in conjunction with the overall network of strategically positioned sensors, which may then be used detect and predict deteriorating posture and potential falls. Also, sensor nodes may be positioned at the wearer's head, as well as one or more locations on the wearer's arms or legs. Such locations may include the shoulders, elbows, and hands/wrists for the arm locations, and the knees, thighs, and feet/ankles for the leg locations. However, these locations are merely exemplary and the disclosed principles may be implemented with any sensor locations that are capable of capturing full body motion capture data. Further, an assistive walking device (e.g., a cane) of the wearer 210 may also be fitted with a geolocation device, such as a GPS tracker 220. Such a GPS tracker 220 on the waking device can assist with determining the geographic location of the wearer 210, if desired, and is discussed in further detail below. Moreover, locations for each sensor node may be selected based on the type of body motion being captured for evaluation. Accordingly, those who are skilled in the pertinent field of art will understand what body locations may thus be used when implementing the 3D body motion mapping provided by the disclosed principles.

When configuring a 3D body movement mapping network, the various sensor nodes 205 may be connected to a central sensor node 220 via a wired or wireless connection to create the sensor network 200. In exemplary embodiments, the central sensor node 220 not only can provide sensor data itself, such as at the body's core location as shown in FIG. 2, but may also collect the data detected by the other sensor nodes 205 in the network 200. In such embodiments, the central sensor node 220 may be wired or wirelessly connected to a computing device, such as a wearable computer, a remote computer, or a mobile device such a tablet computer, where the computing device may be used to evaluate the data gathered by the sensor network 200. In some embodiments, however, the central sensor node 220 itself may have computing capability to analyze the user's movement and posture data captured from all sensor nodes located throughout the patient's body. Alternatively, a central sensor node 220 may not be used to collect data from other sensors 205, and instead each of the sensor nodes 205 may be configured to transmit their gathered data to a computing device, either remote or also carried by the user. In yet other embodiments, each sensor node may have at least limited computing capability to assist in evaluating its own collected data. In any embodiments, the gathered sensor data may also be employed by professional personnel to evaluate user posture, movement, balance, etc., as discussed herein.

Based on the gathered sensor data, the disclosed principles may provide feedback to the wearer to assist in correcting any type of posture or movement issue detected by the network 200. Such detection and feedback may be immediate with regard to when a movement or posture issue is detected. For example, an alarm, which may be a vibration alarm or audible alarm, or any other type of audible, visual, vibrational, textual, or verbal cue, may be used to provide such feedback. Moreover, the feedback may be provided by the sensor nodes 205, 220, or may be provided at one or more other locations on the user's body. More specifically, feedback may be provided at the location(s) of the wearer's body where the problem is occurring or where correction should occur. In other embodiments, the feedback may be provided at a predetermined location, such as via a wristband or necklace worn by the user. Also, the feedback could be provided other than on the user's body, such as to a mobile device carrier by the user, or perhaps by components provided in the user's walking device. Still further, the feedback may not be immediate when detected, and may instead be provided to the user or other person after the fact, as information to be presented to the user once data has been gathered and evaluated. The feedback may also be provided to personnel monitoring the position and movement of the user, such as a doctor of caretaker.

Looking now at FIG. 3, illustrated is a block diagram setting forth the internal components of one embodiment of a sensor node 300 in accordance with the disclosed principles. Specifically, each sensor node may contain a microcontroller 305, 3D inertia sensors 310, and either a wired or wireless connection interface 315 to the central sensor node or computing device for data gathering and/or evaluation.

The inertia sensors 310, which may include a gyroscope, accelerometer, and/or magnetometer, are used to capture the data of body movement and posture change at each sensor node's location to create a 3D map of such movement and posture of the wearer. The microcontroller 305 may provide control of the sensors with each sensor node 300, as well as how the data captured by the sensors 310 is maintained and sent for evaluation. The captured movement and posture data may then be transmitted to the central sensor node via the wired or wireless connection interface 315, or may be transmitted to a dedicated computing device via the interface 315. In wireless interconnected embodiments, the sensor nodes may be interconnected with existing wireless networking technology, such as ZigBee®, Bluetooth, or other wireless technology, either now existing or later developed.

Turning to FIG. 4, illustrated is a block diagram 400 setting forth one embodiment of the interconnection of a central sensor node 405 with an external computing device 430a, 430b. Such a computing device 430a, 430b may be used to gather and/or evaluate the data provided by the network of sensors and collected in the central sensor node 405.

The central sensor node 405 is a sensor node that contains all the functionality of the typical sensor node, such as the sensor node 300 illustrated in FIG. 3. This includes having a microcontroller 410, one or more inertia sensors 415, and sensor network connection interface 420. The central sensor node 405 may also contain local non-volatile memory 425 to store the movement and posture data from its own sensors 415, as well as from each sensor node within the sensor network.

In addition to the wireless connection interface components for interconnecting the central sensor node 405 with other sensor nodes in the network, the central censor node 405 also contains wireless connection with large bandwidth, such as WiFi or Bluetooth, in order to move the data from the local memory storage 425 to a PC or mobile computing device external to the network. Additionally, the central sensor node 405 may contain communication capability via a mobile phone telecommunications network, in which case the movement and posture data can be sent via the mobile phone data network to the external remote computing device.

Referring now to FIG. 5, illustrated is a block diagram 500 setting forth the internal components of one embodiment of a bottom sensor node 500 in accordance with the disclosed principles. As with other sensor nodes in the disclosed sensor network, such a bottom sensor node 500 may also contain a microcontroller 505, 3D inertia sensors 510, and either a wired or wireless connection interface 515 to the central sensor node or computing device for data gathering and/or evaluation.

In addition to such functionality, the bottom sensor node 500 also comprises a weight sensor 520. More specifically, when an assistive walking device, such as a walking stick or cane, is used, the bottom sensor node 500 may be added to the bottom of that assistive walking device. Additionally, such a bottom sensor node 500 may also be employed at the bottom locations of the wearer's body, such as at the bottoms of the wearer's feet. In any of those implementations, the weight sensor 520 provides additional data for the disclosed 3D movement mapping technique, namely the amount of weight applied to the assistive walking device, or each foot of the wearer, at a given point in time.

Looking now at FIG. 6, illustrated is a block diagram 600 of the exemplary central sensor node 405 of FIG. 4 with optional GPS tracking information. As before, the central sensor node 405 comprises a microcontroller 410, one or more inertia sensors 415, a sensor network connection interface 420, and local non-volatile memory 425. The central censor node 405 also includes large bandwidth wireless connection, such as WiFi or Bluetooth, for transferring data from the local memory storage 425 to an external computing device.

In addition to these components and functionality of the central sensor node 405, the disclosed principles may also employ GPS information. More specifically, the GPS information may be provided using a GPS tracker 605 or similar device, which is configured to provide the geographic location of the wearer of the sensor node network. In exemplary embodiments, when the GPS tracker 605 is used, the GPS coordinates of the wearer provided by the tracker may be captured and stored along with the movement and posture data. Such GPS coordinates or other information may be displayed on a display screen in a real time or displayed based on stored data. Moreover, “street images” such as those provide by software like Google® Street View or similar technology may be superimposed with captured data so that the captured movement and posture of the wearer may be interpreted more accurately based on real outdoor walking environments and conditions experienced by the user when movement data has been detected and acquired.

As compared with previously reported inertia sensors used for human body movement detection, the disclosed principles provide a 3D movement mapping network with a number of advantages over conventional approaches. For example, the disclosed 3D network provides information on many more specific body joints, such as head/neck and shoulder regions. This results in a more complete mapping of the wearer's body segments.

Conventional approaches either cover too few joints or use fewer sensors for motion-sensing. In an exemplary embodiment, the disclosed network is comprised of 12 sensor nodes, located at specifically selected locations of the body in order to capture the most complete movement data of the human body with respect to an identification of how the plummet line through the COG moves dynamically within the BOS. Such information has not been provided or reported before in conventional body sensor mapping, regardless of the type of sensors employed in those approaches.

Looking at FIG. 7, illustrated is a diagram of a wearer 700 of a sensor network as disclosed herein, where the specific locations of the sensor nodes 705 are selected for providing such identification. Specifically, in this embodiment, 12 inertia sensor nodes 705 are provided at the following locations on the wearer's body, along with 1 pressure sensor 710 on a cane, and multiple pressure sensors 715 on the wearer's foot areas:
1 head sensor node: external occipital protuberance
2 upper arm sensor nodes: lateral side of deltoid muscle (each side)
1 central sensor node: 5th lumbar vertebral level, approx. center of gravity
2 hip sensor nodes: at lateral side of greater trochanter of femur (each side)
2 thigh sensor nodes: anterior distal thigh proximal to the knee joint (each side)
2 ankle sensor nodes: anterior distal leg proximal to the imaginary line between medial and lateral malleoli (each side).
2 foot sensor nodes: on top of shoes (each side)
1 cane pressure sensor node: at bottom tip of the cane
6 foot pressure sensor nodes: into insoles at bottom of feet (3 each side)

FIG. 8 provides exemplary locations for the 3 pressure sensors 715 that may be included in each of the wearer's feet 800. In addition, a wrist band 720 may be provided for use in, for example, providing GPS information as discussed above, or perhaps for providing movement or pasture feedback to the wearer, also as discussed above.

In the disclosed 3D movement and posture mapping network, the inertia sensors included in each sensor node are combined with pressure sensors such that they will provide not only data of motion but also data of quantitative pressure values resulting from the motion of body segments. In addition, FIG. 9 illustrates how the COG of a wearer 900 change as the wearer changes his positions. Specifically, a central sensor node 905 placed at the wearer's COG location (as identified above) can be seen changing positions compared to when the wearer has his arms at his side, then raises his arms above his head, and then as the wearer bends over forward. Thus, with a sensor network as disclosed herein, clinicians will be able to have a more complete and integrated pictures of the dynamic movement of the COG and BOS of a wearer in real-time. As discussed above, the constantly changing COG of a wearer must be maintained within the constantly changing BOS of the wearer in order to maintain the wearer's stability during his movement. Moreover, clinicians may retrieve data from the network for both real-time time monitoring or for retrograde analysis, based on previously captured user data, of the individual's mobility/posture at a later time.

As shown in FIG. 9, a person's COG changes as the body changes positions (e.g., arms down or up) or posture (e.g., leaning or bending forward). The pressure or the ground reaction force to the changing COG can be consequently different. For example, there will be more pressure on the posterior of both feet when standing with arms-raised, and on the anterior of both feet when standing with upper body bending over. In a wearer's daily life, a constant and controlled change of the COG occurs for routine activities. Accordingly, the disclosed principles assist in ensuring the constantly changing COG of a particular wearer is maintained within a constantly changing BOS to maintain the wearer's stability. Otherwise, a fall may occur due to uncontrolled body movement or uncontrolled change of the COG.

As discussed above, the sensor nodes in the disclosed network have various sensors for measuring various data points. For example, gyroscopes may be used to measure angular movement and accelerometers may be used to determine the direction of the linear movement by sensing acceleration due to gravity. The disclosed principles also employ weight sensor to measure the weight pressure on each foot, and the weight pressure on an assistive walking device if the device is used. The data captured by the sensor nodes throughout the wearer's body may then be used to evaluate:
1) dynamic balance in terms of constant changing of the COG over constant changing of the BOS due to change of direction/position of each body segment in response to the body movement, and
2) dynamic gait in terms of change of gait parameters and body weight pressure on feet (and cane if in cane users).

Movements of body segments, movement of COG, size of BOS, gait parameters (velocity, cadence, stance, and swing time, step and stride length), and gait pressure can be accurately tracked and represented by the inertia sensors and pressure sensors of the disclosed network. With such accurate remote tracking, a wearer's data of balance and gait can thus be traced and evaluated cost effectively either on a real time basis or on recorded basis for later playback. Moreover, an on-screen simulation of a wearer's body movement, posture change, movement of COG over the BOS, and gait pattern during indoor or outdoor activities may also be displayed and evaluated. The recorded data can be stored in the subject's personal history file for progress analysis days, months, or even years later. Original data for a wearer may also be captured, and then a comparison of real-time data with historical data may be used to detect deficiencies in position or movement on an ongoing basis, such as for detecting and predicting deteriorating posture and possible falls. In addition, as detailed above, the data may also be used to provide immediate feedback to the wearer, which assists the wearer in his ongoing movement and posture improvement efforts.

In an exemplary application of the disclosed principles, the size and shape of a wearer's BOS depends on the number of feet standing on the ground, and how those feet are positioned with respect to each other. FIGS. 10A-10D illustrate four examples (A-D) of four different standing positions. These include: (FIG. A) two feet standing parallel, (FIG. B) two feet standing semi-tandem, (FIG. C) a single foot standing, and (FIG. D) two feet standing with a cane. The size of BOS is determined by the number of the person's feet (and the tip of the assistive device (e.g., cane) if used) used in contact with the ground. The wearer's loss of stability (LOS) is around the boundary of the of the BOS area. For example, the LOS may be in anterior-posterior direction (sagittal plane) or the medial-lateral (leftward and rightward) direction (coronal plane). In other cases, the LOS may be in diagonal planes, which can be determined based on these sagittal and coronal planes.

For determining the angle of the COG, the movement of a central sensor node placed, for example, at the L4 vertebral spinous process may indicate the dynamic location of the COG plummet line over the center of the BOS. In a static upright standing (baseline) position, the plummet line (COG over BOS) should pass through both the central node and the center of the BOS, but the body will still sway around or deviate away from the vertical plummet line more or less depending on the individual's balance ability. In other words, when the body sways or changes position, the line between the COG (central sensor node) and the center of the BOS will deviate at an angle away from the original vertical plummet line. That angle may be defined as the COG angle.

FIG. 11 illustrates the relationship of the COG angle as determined from the detected COG and BOS provided by the disclosed sensor network. The baseline COG angle (A_{B}) indicates the largest body sway angle in a certain direction at quiet standing, while the maximal value of the COG angle (A_{M}) can be reached when the COG (or the central sensor node) is exactly perpendicular over the boundary of the LOS in that direction (to the left or right). The difference between the A_{B} and the A_{M} indicates the deviation of the COG angle (A_{D}) a person can move without falling. Put together, the COG angle A_{B} is a good indicator for a person's stability, and the COG angle A_{D} is a person's natural dynamic deviation during normal gait. The bigger the A_{B} is, the less room for A_{D} before A_{D} reaches the maximum angle (A_{M}). Thus, the disclosed sensor network may use this geometric approach to determine that a person will typically fall when his COG angle is over its maximal or beyond the boundary of the BOS.

Additionally, the detection of the LOS will typically differ for each wearer of the disclosed sensor network, and will also change over time due to the dynamic nature of the wearer's COG and BOS as the wearer moves. Thus, a wearer of the body sensor network (e.g., a patient) may have a baseline or threshold established by having their posture, gait, or other body positions and movements initially monitored and recorded using the disclosed sensor network. That baseline data may then be stored and compared against the wearer's later body positions and movements, as continually detected by the disclosed sensor network, to detect degradation in one or more positions or movements over time, and additionally detect the potential for falls or other detrimental results of the wearer's current and ongoing body positioning and movement.

With the ability of a system or method implemented in accordance with the disclosed principles to obtain complete and detailed balance, gait, and posture information, the disclosed 3D movement and posture mapping network is a valuable tool for physicians to build up a patient's baseline balance, gait, and posture profile, and perform periodic evaluations of the patient's profile. In addition, the obtained individualized information may be employed for the individual patient to use in any environment, even when the user is alone at home. In such embodiments, immediate feedback may be employed to assist the individual in correcting any movement or posture deficiencies, as well as to collect data for later review by the individual in addition to any clinical review. Original data for a wearer may also be compared against real-time data to detect deficiencies in position or movement on an ongoing basis, such as for detecting and predicting deteriorating posture and possible falls.

FIGS. 12A-12C illustrate plots, measured in inches, of an exemplary wearer's COG over BOS in varying stages of stability. In these exemplary plots, the BOS is represented by the origin of the graph, while the COG is represented as traced movement of the COG with respect to the BOS over an evaluation period. When the wearer is generally stable, the plot of his COG over BOS is illustrated as in FIG. 12A, where the deviation of the traced COG with respect to the BOS is quite small. FIG. 12B demonstrates an increasingly unstable COG with respect to the wearer's BOS, as illustrated by the increased deviation of the traced COG with respect to the BOS. Moreover, the direction(s) of the detected instability may also be determined using the plot(s) created by the disclosed sensor network. FIG. 12C demonstrates an even further increased unstable COG with respect to the wearer's BOS, as illustrated by the further increased deviation of the traced COG with respect to the BOS.

In this example, the LOS for this particular wearer may have been determined to be when the deviation of the wearer's COG over his BOS exceeds approximately 1 inch in any direction. Such determination may differ for each wearer, and therefore a baseline or threshold measurement(s) may be obtain initially, and recorded for individualized comparison for each wearer. For the particular wearer in this embodiment, FIG. 12B would inform the wearer or monitoring personnel that the wearer is at his limit of stability, and therefore is on the verge of losing his stability. Additionally, FIG. 12C would inform the wearer or monitoring personnel that the wearer has exceed his limit of stability, and thus his either already lost his stability will likely do so in the immediate future.

Looking finally at FIG. 13, illustrated is a flow diagram 1300 setting forth an exemplary process for determining and predicting the deteriorating gait a wearer for a sensor network as disclosed herein. At step 1301, a wearable sensor network in accordance with the disclosed principles is donned by the subject wearer. As detailed, above the sensor data from all the strategically placed sensors is captured, and the results stored. At step 1302, that stored data is analyzed by professionals, and then at step 1303 results of the data analysis is used to create a baseline or threshold of his LOS, which is comprised of the wearer's detected COG over his BOS. That baseline data is then used to create a personalized walking pattern for the particular wearer, at step 1304, which is based on the position and movement of each part of his body during the initial data capture. As a given period of time elapses, the sensor network captures current data based on the wearer's current gait. Then, at step 1305, a pattern recognition algorithm may be used to determine deviations in the wearer's current gait as compared to the wearer's originally capture gait pattern. In exemplary embodiments and as discussed above, the algorithm may be selected to determine deviations of the wearer's COG with respect to his BOS. However, in other embodiments, other data patterns may be detected and compared by the algorithm used, and thus the disclosed principles are not limited to any particular algorithm or data.

At step 1306, the process determines if there is a measureable change in the current walking pattern when compared to the baseline pattern, again using the dynamic change in both COG over BOS of the wearer over time. The amount of measureable change may be any predetermined amount, and will differ between wearers. If the detected pattern change exceeds the predetermined threshold, the moves to step 1307 where a doctor or other monitoring personnel may be notified of the issue. The notification may be based on the wearer's walking pattern deteriorating beyond a predetermined (e.g., “safe”) point, or may even be based on the newly detected walking pattern demonstrating that the wearer has fallen. The doctor or other personnel may then address the issue with the wearer, for example, providing a walking assistance device or therapy or other procedure to address the deficiency.

If at step 1306 any detected changes in the walking pattern of the wearer did not exceed the predetermined threshold, the process moves to step 1308 where the newly gathered data may be analyzed and the results used to predict the deterioration of the wearer's walking pattern. If that analysis determines that there is a deteriorating trend, the process then determines at step 1309 whether that trends exceeds a predetermined limit, and thus a trend issue is present. If such an issue is present, the process may then move to the notification of a doctor or other personnel to address the trend issue with the wearer in whatever manner can help the wearer correct the deficiency. If the current walking pattern does not raise a trend issue, the process moves to step 1310, where new and current data of the walking pattern of the wearer is gathered. Then the process reverts back to the beginning, where the newly captured data is stored and then analyzed for changes in walking pattern.

Although the process of FIG. 13 illustrates a process for analyzing the walking pattern of a wearer, the same or similar process may be used to analyze different position, posture, or movement of a wearer. Additionally, the specific steps included in the process illustrated in FIG. 13 are merely exemplary, and other processes within the broad scope of the disclosed principles may include a greater or lesser number, or even different, steps than those illustrated.

While various embodiments in accordance with the principles disclosed herein have been described above, it should be understood that they have been presented by way of example only, and not limitation. Thus, the breadth and scope of this disclosure should not be limited by any of the above-described exemplary embodiments, but should be defined only in accordance with any claims and their equivalents issuing from this disclosure. Furthermore, the above advantages and features are provided in described embodiments, but shall not limit the application of such issued claims to processes and structures accomplishing any or all of the above advantages.

Additionally, the section headings herein are provided for consistency with the suggestions under 37 C.F.R. 1.77 or otherwise to provide organizational cues. These headings shall not limit or characterize the invention(s) set out in any claims that may issue from this disclosure. Specifically and by way of example, although the headings refer to a “Technical Field,” the claims should not be limited by the language chosen under this heading to describe the so-called field. Further, a description of a technology in the “Background” is not to be construed as an admission that certain technology is prior art to any embodiment(s) in this disclosure. Neither is the “Summary” to be considered as a characterization of the embodiment(s) set forth in issued claims. Furthermore, any reference in this disclosure to “invention” in the singular should not be used to argue that there is only a single point of novelty in this disclosure. Multiple embodiments may be set forth according to the limitations of the multiple claims issuing from this disclosure, and such claims accordingly define the embodiment(s), and their equivalents, that are protected thereby. In all instances, the scope of such claims shall be considered on their own merits in light of this disclosure, but should not be constrained by the headings set forth herein.

1. A system for evaluating movement of a human body, the system comprising:
a central sensor node positioned substantially at the center of gravity of the body;
a network of sensors dispersed about predetermined locations on the body and configured to gather data regarding positions of the predetermined locations during movement of the body's center of gravity with respect to the body's base of support;
a computing device configured to receive sensor data from the central sensor node and the network of sensors, and based on the received data:
detect balance deficiencies of the body based on positions of the body's center of gravity in relation to corresponding positions of the body's base of support during movement of the body, and
evaluate the gathered data to determine unfavorable positions of one or more of the predetermined locations during detected balance deficiencies; and
a feedback device configured to provide feedback when one or more of the predetermined locations of the body moves into one or more corresponding unfavorable positions.

2. A system in accordance with claim 1, wherein the network of sensors comprise one or more inertia sensors and pressure sensors.

3. A system in accordance with claim 1, wherein one or more of the sensors is configured to capture movement of joint-based body parts to determine dynamic change of posture of the user's body during ambulation.

4. A system in accordance with claim 1, wherein the predetermined locations for the network of sensors comprise at least 1 head sensor, 2 upper arm sensors, 2 hip sensors, 2 thigh sensors, 2 ankle sensors, and 2 foot sensors.

5. A system in accordance with claim 1, wherein the predetermined locations for the network of sensors further comprise pressure sensors at the bottom of the user's feet.

6. A system in accordance with claim 1, wherein the system further comprises a pressure sensor at a bottom tip of a walking device configured for use by the user during ambulation, the computing device further configured to receive sensor date from the walking device pressure sensor.

7. A system in accordance with claim 1, wherein the central sensor node comprises the computing device.

8. A system in accordance with claim 1, wherein the network of sensors comprises one or more of 3D gyroscopes, 3D accelerometers, and 3D magnetometers.

9. A system in accordance with claim 1, wherein one or more of the network of sensors and central sensor node comprises wireless data transmission capabilities for transmitting the sensor data.

10. A system in accordance with claim 1, wherein the computing device is wearable on the body.

11. A system in accordance with claim 1, wherein the computing device is remote from the body.

12. A system in accordance with claim 1, wherein the computing device is a mobile computing device configured to be carried by a user owning the body.

13. A system in accordance with claim 1, wherein the computing device is further configured to provide a visual display of the predetermined locations during movement of the body based on the received sensor data.

14. A system in accordance with claim 1, wherein the provided feedback comprises one or more of an audible or vibrational cue.

15. A system in accordance with claim 1, wherein the feedback device is configured to provide the feedback at the one or more predetermined locations when said one or more predetermined locations moves into one or more corresponding unfavorable positions.

16. A method for evaluating movement of a human body, the system comprising:
detecting the body's center of gravity using data gathered from a central sensor;
gathering data from a network of sensors positioned at predetermined locations on the body during movement of the body's center of gravity with respect to the body's base of support;
detecting, based on the data gathered from the central sensor and the network of sensors, balance deficiencies of the body based on positions of the body's center of gravity in relation to corresponding positions of the body's base of support during movement of the body;
evaluating the gathered data to determine unfavorable positions of one or more of the predetermined locations during detected balance deficiencies; and
providing feedback when one or more of the predetermined locations of the body moves into one or more corresponding unfavorable positions.

17. A method in accordance with claim 16, wherein the network of sensors comprise one or more inertia sensors and pressure sensors.

18. A method in accordance with claim 16, wherein one or more of the sensors is configured to capture movement of joint-based body parts to determine dynamic change of posture of the user's body during ambulation.

19. A method in accordance with claim 16, wherein the predetermined locations for the network of sensors comprise at least 1 head sensor, 2 upper arm sensors, 2 hip sensors, 2 thigh sensors, 2 ankle sensors, and 2 foot sensors.

20. A method in accordance with claim 16, wherein the predetermined locations for the network of sensors further comprise pressure sensors at the bottom of the user's feet.

21. A method in accordance with claim 16, wherein the method further comprises gathering data from a pressure sensor located at a bottom tip of a walking device configured for use by the user during ambulation.

22. A method in accordance with claim 16, wherein the central sensor node comprises a computing device configured to provide the detecting and evaluating.

23. A method in accordance with claim 16, wherein the network of sensors comprises one or more of 3D gyroscopes, 3D accelerometers, and 3D magnetometers.

24. A method in accordance with claim 16, wherein one or more of the network of sensors and central sensor node comprises wireless data transmission capabilities for transmitting the sensor data.

25. A method in accordance with claim 16, wherein the detecting and evaluating is provided using a computing device wearable on the body.

26. A method in accordance with claim 16, wherein the detecting and evaluating is provided using a computing device remote from the body.

27. A method in accordance with claim 16, wherein the detecting and evaluating is provided using a mobile computing device configured to be carried by a user owning the body.

28. A method in accordance with claim 16, further comprising providing a visual display, on a computing device, of the predetermined locations during movement of the body based on the received sensor data.

29. A method in accordance with claim 16, wherein providing feedback comprises providing one or more of an audible or vibrational cue.

30. A method in accordance with claim 16, wherein providing feedback further comprises providing feedback at the one or more predetermined locations when said one or more predetermined locations moves into one or more corresponding unfavorable positions.
